(19) United States
(12) Patent Application Publication
(10) Pub. No.: US 2026/0238218 A1
(43) Pub. Date: Aug. 13, 2026

(54) CIRCUITRY FOR MEASUREMENT OF ELECTROCHEMICAL CELLS

(71) Applicant: Cirrus Logic International Semiconductor Ltd., Edinburgh (GB)

(72) Inventors: Peter HSU, Edinburgh (GB); Gordon J. BATES, Edinburgh (GB); Vivek SARAF, Edinburgh (GB); John PRIESTLEY, Edinburgh (GB); Ivan PERRY, Edinburgh (GB); Yu TAMURA, Tokyo (JP); James WELLS, Edinburgh (GB); Louis FREW, Edinburgh (GB); Paul WILSON, Edinburgh (GB)

(73) Assignee: Cirrus Logic International Semiconductor Ltd., Edinburgh (GB)

(21) Appl. No.: 19/155,418

(22) PCT Filed: Mar. 6, 2024
(86) PCT No.: PCT/GB2024/050592

§ 371 Date: Aug. 11, 2025

Related U.S. Application Data
(63) Continuation of application No. 18/192,257, filed on Mar. 29, 2023, now Pat. No. 12,308,852.
(60) Provisional application No. 63/450,456, filed on Mar. 7, 2023.
(60) Provisional application No. 63/603,732, filed on Nov. 29, 2023.

(51) Int. Cl. H03M 1/12; H03M 1/10

(52) U.S. Cl. CPC H03M 1/121; H03M 1/1023

(57) ABSTRACT

A circuitry for processing an analyte signal obtained from an electrochemical cell comprising a first electrode and a second electrode, comprises: a drive circuitry comprising a drive amplifier configured to apply a stimulus to the first electrode; a measurement circuitry comprising a measurement amplifier configured to measure a response at the second electrode of the electrochemical cell to the stimulus; an analog-to-digital converter (ADC) configured to output a digital output in dependence on the measured response; and a control circuitry operable to transition the circuitry between a measurement mode and a diagnostic mode, wherein: in the measurement mode, the measurement circuitry is configured with a first bandwidth for measuring an analyte at the second electrode of the cell; and in a diagnostic mode the measurement circuitry is configured with a second bandwidth for measuring an impedance of the second electrode of the cell, wherein the second bandwidth is greater than the first bandwidth.

### TECHNICAL FIELD

The present disclosure relates to circuitry for measuring characteristics in electrochemical sensors.

### BACKGROUND

Electrochemical sensors are widely used for the detection of one or more particular chemical species, analytes, as an oxidation or reduction current (or voltage in the case of potentiometric sensors). Such sensors comprise an electrochemical cell, consisting of two or more electrodes configured for contact with an analyte whose concentration is to be ascertained. Such sensors also comprise circuitry for driving one or more of the electrodes and for measuring a response at one or more of the electrodes. The sampled response signal can be processed to determine a concentration of an analyte.

Electrochemical cells are often integrated into wearable health monitors, such as continuous glucose monitors. When such devices are battery powered, it is desirable for the sensor to be as small as possible and use as little power as possible.

Conventional measurement circuitry used in electrochemical sensors may have certain bandwidth limitations. For example, transimpedance amplifiers may operate efficiently at low frequencies, but can be slow when operating at high frequencies. Conversely, current conveyors may operate well over larger bandwidths but can suffer from accuracy issues due lack of feedback in their output stages.

### SUMMARY

According to an aspect of the disclosure, there is provided circuitry for processing an analyte signal obtained from an electrochemical cell comprising a first electrode and a second electrode, the circuitry comprising: drive circuitry comprising a drive amplifier configured to apply a stimulus to the first electrode; measurement circuitry comprising a measurement amplifier configured to measure a response at the second electrode of the electrochemical cell to the stimulus; an analog-to-digital converter (ADC) configured to output a digital output in dependence on the measured response; and control circuitry operable to transition the circuitry between a measurement mode and a diagnostic mode, wherein: in the measurement mode, the measurement circuitry is configured with a first bandwidth for measuring an analyte at the second electrode of the cell; and in a diagnostic mode the measurement circuitry is configured with a second bandwidth for measuring an impedance of the second electrode of the cell, wherein the second bandwidth is greater than the first bandwidth.

The control circuitry may be configured to transition the measurement circuitry between the measurement mode and the diagnostic mode by adjusting a first bias signal applied to the measurement amplifier.

One or both of the measurement amplifier and the drive amplifier may comprises a multistage amplifier. The multistage amplifier may comprise: a first amplifier stage comprising a first stage input and a first stage output, the first amplifier stage biased with a first bias signal; and a second amplifier stage having a second stage input coupled to first stage output and a second stage output, the second amplifier stage biased with a second bias signal. Transitioning the circuitry between the measurement mode and the diagnostic mode may comprise adjusting the first bias signal and the second bias signal.

The first and second bias signals may be adjusted to maintain a constant DC bias at the output of the second amplifier stage.

Transitioning the circuitry from the diagnostic mode to the measurement mode may comprise, during a first time period, reducing a level of the second bias signal; and in a second time period, reducing a level of the first bias signal, the second time period occurring after the first time period.

The first and second time period may be separated by an intermediate time period. During the intermediate time period, the control circuitry may be configured to adjust a compensation scheme of the multistage amplifier.

The compensation scheme may be adjusted to reduce capacitive loading on the first amplifier stage during the intermediate time period.

The compensation scheme may be adjusted to increase a response of the first amplifier stage during the intermediate time period.

Transitioning the circuitry from the measurement mode to the diagnostic mode may comprise: increasing a level of the second bias signal; and increasing a level of the first bias signal, the levels of the first and second bias signals increased substantially simultaneously.

The measurement circuitry may comprise: a first signal path between the second electrode of the electrochemical cell and a first input of the ADC, the first signal path comprising a first gain stage configured to convert the analyte signal to a first analog signal; a second signal path between the first electrode and the first input of the ADC, the second signal path comprising a second gain stage configured to convert the analyte signal to a second analog signal, first gain stage having a higher bandwidth than the second gain stage; and switching circuitry configured to selectively couple the second electrode to the first input of the ADC. The control circuitry may be configured to control the switching circuitry to couple the second electrode to the first input of the ADC to transition the circuitry from the measurement mode to the diagnostic mode.

The first gain stage may comprise a current conveyor and the second gain stage may comprise a transimpedance amplifier (TIA). The TIA may comprise a feedback resistor coupled between the second electrode and the first input of the ADC, wherein selectively coupling the first electrode to the first input of the ADC circuit may comprise bypassing the feedback resistor.

In the measurement mode, the drive circuitry may be configured with the first bandwidth; and in the diagnostic mode, the drive circuitry is configured with the second bandwidth.

The control circuitry may be operable to transition the measurement circuitry between the measurement mode and the diagnostic mode by adjusting a second bias signal applied to the drive amplifier.

The drive circuitry may comprise a digital-to-analog converter (DAC) configured to apply the second bias signal to the drive circuitry.

The DAC may be configurable in the measurement mode and the diagnostic mode. Configuring the DAC between the measurement mode and the diagnostic mode may comprise adjusting a sampling rate of the DAC.

The ADC may be configurable in the measurement mode and the diagnostic mode. Configuring the ADC between the measurement mode and the diagnostic mode may comprise adjusting a sampling frequency of the ADC. The sampling frequency in the measurement mode may be lower than the sampling frequency in the diagnostic mode.

The ADC may be configurable in the measurement mode and the diagnostic mode. Configuring the ADC between the measurement mode and the diagnostic mode may comprise adjusting an ADC supply voltage provided to the ADC.

In the measurement mode, the stimulus may comprises a DC component, whereas in the diagnostic mode, the stimulus may comprise both an DC component and an AC component.

The AC component of the stimulus may comprise a sinusoid, a step function, or an impulse function.

In the measurement mode, the circuitry may be configured to determine an analyte concentration of the analyte at the second electrode.

In the diagnostic mode, the circuitry may be configured to determine a status of the second electrode in dependence on the measured impedance. The status may comprise one or more of the following: a state of health of the electrochemical cell; a state of degradation of the second electrode; and a fault at the electrochemical cell.

The control circuitry may be operable to configure the circuitry in the measurement mode or the diagnostic mode in response to a command from an external controller.

The control circuitry may be operable to transition the circuitry between the measurement mode and the diagnostic mode after a predetermined time period since a last transition between the measurement mode and the diagnostic mode.

According to another aspect of the disclosure, there is provided circuitry for processing an analyte signal obtained from an electrochemical cell comprising a first electrode and a second electrode, the circuitry comprising: drive circuitry comprising a drive amplifier configured to apply a stimulus to a first electrode; measurement circuitry comprising a measurement amplifier configured to measure a response at the second electrode; wherein one or both of the measurement amplifier and the drive amplifier comprises a multistage amplifier. The multistage amplifier may comprise: a first amplifier stage comprising a first stage input and a first stage output, the first amplifier stage biased with a first bias signal; and a second amplifier stage having a second stage input coupled to first stage output and a second stage output, the second amplifier stage biased with a second bias signal.

The circuitry may further comprise control circuitry operable to transition the circuitry between a measurement mode and a diagnostic mode. Transitioning the circuitry between the measurement mode and the diagnostic mode may comprise adjusting the first bias signal and the second bias signal.

According to another aspect of the disclosure, there is provided circuitry for processing an analyte signal obtained from an electrochemical cell, the circuitry comprising: a first signal path between a first electrode of the electrochemical cell and a first input of an analog-to-digital converter (ADC) circuit, the first signal path comprising a first gain stage configured to convert the analyte signal to a first analog signal; a second signal path between the first electrode and a second input of the ADC circuit, the second signal path comprising a second gain stage configured to convert the analyte signal to a second analog signal; and switching circuitry configured to selectively couple the first electrode to the first input of the ADC circuit.

The second gain stage may have a higher accuracy than the first gain stage.

The first gain stage may have a higher output impedance than the second gain stage.

The second gain stage may comprise a transimpedance amplifier. The transimpedance amplifier may comprise: a feedback resistor coupled between the first electrode and the first input of the ADC circuit, wherein selectively coupling the first electrode to the first input of the ADC circuit comprises bypassing the feedback resistor.

Bypassing the feedback resistor may cause the gain stage to operate as a unity buffer between the first electrode and the first input of the ADC circuit.

The switching circuitry may be configured to switch circuitry between a first mode and a second mode. In the first mode, the circuitry may operate as a current conveyer. In the second mode, the circuitry may operate as a transimpedance amplifier (TIA).

The first gain stage may comprise a current conveyer.

The circuitry may further comprise the ADC circuit. The ADC circuit may comprise: a first ADC and a second ADC. The first ADC may be configured to convert the first analog signal to a first digital signal. The second ADC may be configured to convert the second analog signal to a second digital signal.

The first ADC may have a higher bandwidth than the second ADC.

The circuitry may further comprise a digital correction module configured to apply a correction factor to the first digital signal and output a corrected first digital signal. The correction factor may be configured to correct an error introduced by the first signal path.

The correction factor may be configured to correct one or more of: a DC offset in the first analog signal; a gain of the first analog signal; and distortion in the first analog signal.

The circuitry may further comprise memory for storing the correction factor.

The correction factor may be calculated based on a comparison between the first analog signal and the second analog signal.

During a calibration phase, the circuitry may be configured to: apply a calibration stimulus at the first electrode; measure the first analog signal responsive to the calibration stimulus; measure the second analog signal responsive to the calibration stimulus; and determining the correction factor based on the first and second analog signals.

The calibration stimulus may comprise one of: one or more DC signals; a swept DC signal; a combination of DC and AC signals (e.g., a chirp).

The circuitry may be configured to: monitor the analyte signal, the first analog signal and the second analog signal; and update the correction factor based on the monitored analyte signal, the first analog signal, and the second analog signal. The correction factor may be updated periodically or continuously.

According to another aspect of the disclosure, there is provided a system comprising: the circuitry of any one of the preceding claims; the electrochemical cell.

The electrochemical cell may comprise a counter electrode; the first electrode is a first working electrode of the electrochemical cell.

The electrochemical cell may comprise one or more second working electrodes.

The electrochemical cell may comprise an anode and a cathode. The first electrode may be the cathode.

According to another aspect of the disclosure, there is provided an analyte sensor comprising any of the circuitry or the systems described above.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### BRIEF DESCRIPTION OF DRAWINGS

Embodiments of the present disclosure will now be described by way of non-limiting examples with reference to the drawings, in which:

FIG. 1 illustrates a schematic diagram and electrical equivalent circuit for a three-electrode electrochemical cell;

FIG. 2 illustrates a schematic diagram and electrical equivalent circuit for a two-electrode electrochemical cell;

FIG. 3A is a schematic diagram of an example prior art drive and measurement circuit;

FIG. 3B is a schematic diagram of an example implementation of the drive and measurement circuit of FIG. 3A;

FIG. 3C is a schematic diagram of an example implementation of the drive and measurement circuit of FIG. 3A;

FIG. 4 is a schematic diagram of a measurement circuit for measuring characteristics of an electrochemical cell;

FIG. 5 is a schematic diagram of a multistage amplifier;

FIG. 6 is a timing diagram for transitioning bias currents for stages of the multistage amplifier of FIG. 5;

FIG. 7 is a schematic diagram of a measurement circuit which is a variation of the circuit shown in FIG. 4;

FIG. 8 is a schematic diagram of a measurement circuit for measuring characteristics of an electrochemical cell;

FIG. 9 is a schematic diagram of a measurement circuit which is a variation of the circuit shown in FIG. 8;

FIG. 10 is a block diagram of a correction module for correcting a digital output signal generated by the measurement circuit of FIG. 8; and

FIG. 11 is a schematic diagram of a measurement circuit for measuring characteristics of an electrochemical cell comprising two working electrode.

### DESCRIPTION OF EMBODIMENTS

Electrochemical sensors are widely used for the detection of one or more particular chemical species, analytes, as an oxidation or reduction current. Such sensors comprise an electrochemical cell, consisting of two or more electrodes configured for contact with an analyte whose concentration is to be ascertained. Such sensors also comprise circuitry for driving one or more of the electrodes and for measuring a response at one or more of the electrodes. Batteries also comprise one or more electrochemical cells which typically consist of two or more electrodes (e.g., an anode and a cathode) configured for contact with a conductive electrolyte. Characteristics of batteries may be ascertained using drive and measurement circuitry similar to that used for characterising electrochemical cells in electrochemical sensors.

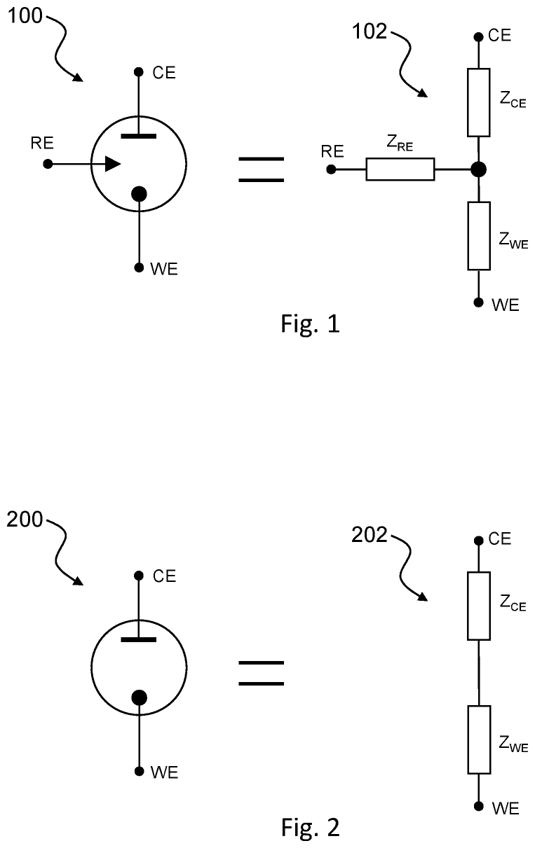

FIG. 1 is a schematic diagram of an electrochemical cell 100 comprising three electrodes, namely a counter electrode CE, a working electrode WE and a reference electrode RE. FIG. 1 also shows an equivalent circuit 102 for the electrochemical cell comprising a counter electrode impedance ZCE, a working electrode impedance ZWE and a reference electrode impedance ZRE.

FIG. 2 is a schematic diagram of another example electrochemical cell 200 comprising two electrodes, namely a counter electrode CE and a working electrode WE. The electrochemical cell 200 varies for the cell 100 with the omission of the reference electrode RE. FIG. 2 also shows an equivalent circuit 102 for the electrochemical cell 200 comprising a counter electrode impedance ZCE and a working electrode impedance ZWE.

In some embodiments, the working electrode WE comprises an assay or chemical of interest. For example for the analysis of glucose as an analyte, the working electrode may comprise a layer of glucose oxidase. The counter electrode CE is provided to form an electrical or ohmic connection with the working electrode WE. Optionally, the reference electrode is provided, which is typically a sensing point between the working electrode WE and the counter electrode CE, allowing independent measurement of the potential associated with each of the working and counter electrodes WE. CE, rather than just measuring a potential difference between the counter and working electrodes CE, WE.

To determine a characteristic of either of the electrochemical cells 100, 200, and therefore an analyte concentration, a bias voltage is applied at the counter electrode CE and a current at the working electrode WE is measured. Feedback is used to set the voltage VRE at the reference electrode RE to be equal to a bias voltage VBIAS1 (as is explained in more detail below) A current IWE at the working electrode WE is then measured. As the resistance in the cell 100 increases, the current measured at the working electrode WE decreases. Likewise, as the resistance in the cell 100 decreases, the current measured at the working electrode WE increases. Thus the electrochemical cell 100 reaches a state of equilibrium where the voltage drop between the reference electrode RE and the working electrode WE is maintained constant. Since the bias voltage at the counter electrode CE and the measured current at WE are known, a characteristic of the analyte contained in the cell 100 can be ascertained.

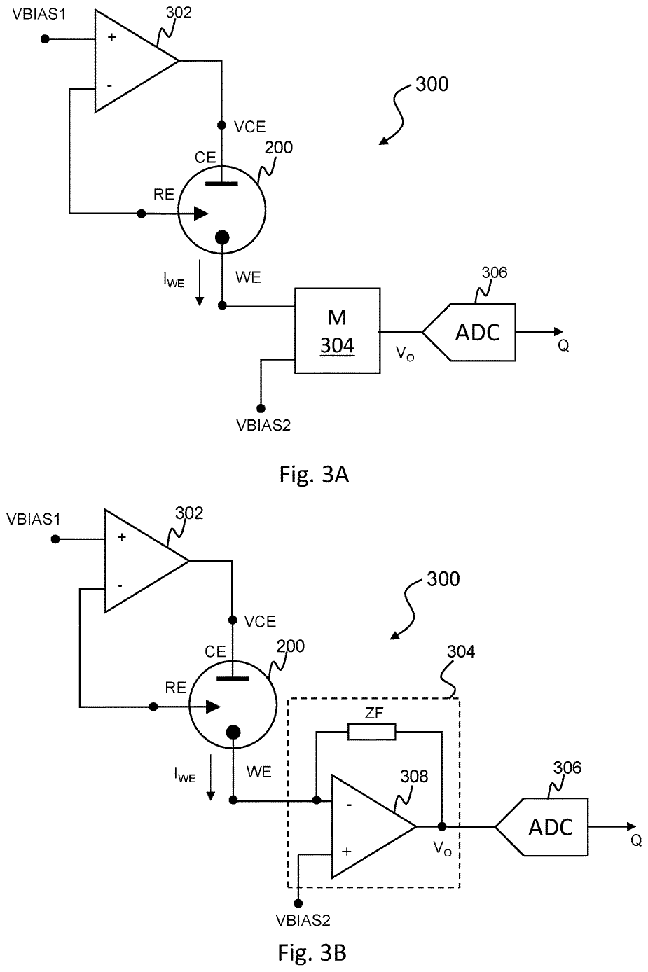

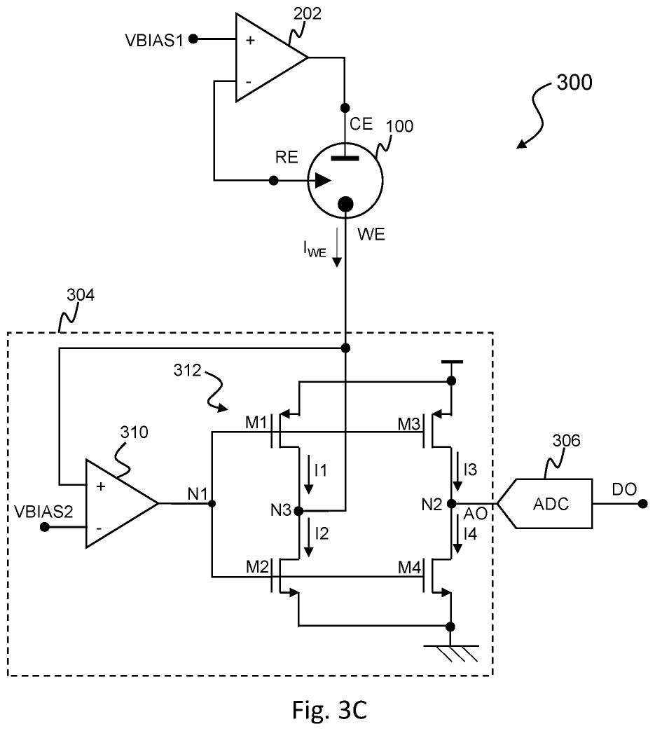

FIG. 3A illustrates an example known drive and measurement circuit 300 which is configured to implement the above explained cell characterisation, specifically for measuring an analyte concentration in the electrochemical cell 100 shown in FIG. 2. The circuit 300 comprises a first amplifier 302 and a measurement circuit 304. Each of the first amplifier 302 and the measurement circuit 304 may comprise one or more op-amps. A non-inverting input of the first amplifier 302 is coupled to a bias voltage VBIAS1 which may be generated by a digital-to-analog converter DAC (not shown). An inverting input of the first amplifier 302 is coupled to the reference electrode RE. An output of the first amplifier 302 is coupled to the counter electrode CE and configured to drive the counter electrode CE with a counter electrode bias voltage VCE. The counter electrode bias voltage VCE applied at the counter electrode CE by the first amplifier 202 is proportional to the difference between the bias voltage VBIAS1 and the voltage VRE at the reference electrode RE.

The measurement circuit 304 is coupled between the working electrode WE and an analog-to-digital converter (ADC) 306. The measurement circuit 304 is operable to output to the ADC 306 a signal proportional to the current flowing from the working electrode WE. The ADC 306 then converts the signal output from the measurement circuit 304 to a digital output signal Q which represents the current flowing from the working electrode WE.

The measurement circuit 304 is typically implemented as a transimpedance amplifier or a current conveyor.

FIG. 3B illustrates an example implementation of the drive and measurement circuit 300, the measurement circuit 304 implemented as a transimpedance amplifier comprising a second amplifier 308. An inverting input of the second amplifier 308 is coupled to the working electrode WE and a non-inverting input of the second amplifier 308 is coupled to a fixed bias voltage VBIAS2, for example ground GND. A feedback impedance ZF is coupled between the non-inverting input and an output of the second amplifier 308. As such, the second amplifier 308 operates as a transimpedance amplifier. The second amplifier 308 is thus operable to output a voltage VO which is proportional to the current IWE at the working electrode WE. The output voltage VO is then provided to the analog-to-digital converter (ADC) 306 which outputs a digital output Q which represents the current IWE at the working electrode WE.

To bias the counter electrode CE, and therefore the electrochemical cell 100, at different voltages, the bias voltage VBIAS1 provided to the first amplifier 302 may be adjusted. The bias voltage VBIAS1 may be adjusted between a reference voltage (e.g. ground or zero volts) and the supply voltage VDD. With the non-inverting input of the second amplifier 204 is set at VDD/2, a positive bias may be applied to the cell 100 by maintaining the bias voltage VBIAS above VDD/2. Likewise, a negative bias may be applied to the cell 100 by maintaining the bias voltage VBIAS below VDD/2. Additionally or alternatively to varying the bias voltage VBIAS1, the reference bias voltage VBIAS2 may be adjusted to set the voltage at the working electrode WE, and therefore the electrochemical cell.

In the example shown in FIG. 3B, the cell 100 is in the form of an amperometric sensor. As such, the stimulus applied to the cell 100 is a voltage stimulus and the measured response is a current. In other embodiments, a potentiometric sensor or battery may be characterised by applying a current stimulus and measuring a voltage response of the cell(s) comprised in the sensor or battery. In either case, an ADC, such as the ADC 306, is provided which samples an output signal to generate a digital output Q comprising a plurality of samples of the output signal.

An advantage of the circuit 300 of FIG. 3B is its relative simplicity. Measurement is implemented using a single amplifier (the second amplifier 204) in combination with the feedback resistor RF. In addition, the use of feedback to set voltages at the counter, working and reference electrode CE, WE, RE of the electrochemical cell 100 minimises gain error through the signal path. This improves accuracy of the circuit 300, particularly for low bandwidth or near DC measurements, for example where changes in the working electrode current IWE are relatively slow. Such low bandwidth measurements may, for example, be in the region of 0.1 to 10 Hz, 0 to 10 Hz, or 0 to 1 Hz.

A disadvantage of the circuit 300 of FIG. 3B is that the working electrode WE of the electrochemical cell 100 is driven by the feedback resistor RF. Since the electrochemical cell 100 represents a complex capacitive load, this can have implications for frequency and/or magnitude of signals that can be driven across the cell 100. For example, driving a change in voltage at the working electrode WE at high frequencies may be slow when compared to driving such a change at low frequencies.

FIG. 3C illustrates another example implementation of the drive and measurement circuit 300, the measurement circuit 304 implemented as a current conveyor

In this example, the measurement circuitry 304 implements a second generation current conveyor (CCII) although other current conveyor topologies could be implemented without departing from the scope of the present disclosure. The measurement circuit 302 comprises a second amplifier 310 (e.g., an operational amplifier) and current mirror circuitry 312 comprising first, second, third and fourth transistors M1, M2, M3, M4. In this example, the transistors M1:M4 are MOSFETs. Specifically, the first and third transistors M1, M3 are PMOS devices and the second and fourth transistors M2, M4 are NMOS devices.

The second amplifier 310 comprises a non-inverting input coupled to the working electrode WE, an inverting input coupled to a reference voltage VBIAS2 and an output coupled to a first (intermediate) node N1.

Gates of each of the first, second, third and fourth transistors M1:M4 are coupled to the first node N1 and therefore the output of the second amplifier 310. Drains of the first and third transistor M1, M3 are coupled to a supply voltage VDD. Sources of the first and third transistors M1, M3 are coupled to drains of the second and fourth transistors M2, M4, respectively. Sources of the second and fourth transistors M2, M4 are coupled to a ground reference voltage (GND). The source of the third transistor M3 and the drain of the fourth transistor M4 are coupled at a second (output) node N2 to an input of the ADC 306. The source of the first transistor M1 and the drain of the second transistor M2 are coupled at a third (feedback) node N3 to the working electrode WE. As such, a feedback path is provided between the third node N3 and the non-inverting input of the second amplifier 310. The second amplifier 310 is thus arranged as a unity gain amplifier or buffer amplifier. The first and second transistors M1, M2 operate as transconductors which generate first and second currents 11, 12 respectively. The working electrode current IWE is equal to the difference between the first and second currents (IWE=I2−I1). The first and second transistors M1, M2 act as input reference devices of a current mirror. The third and fourth transistors M3, M4 operate as output devices of the current mirror. The first current 11 is mirrored to a third current 13 generated by the third transistor N3. The second current 12 is mirrored to a fourth current 14 generated by the fourth transistor N4.

During operation, the working electrode current IWE is provided to the second amplifier 310 and this current IWE is amplified by unity and therefore buffered to the first node N1. During operation, the second amplifier 310 amplifies the difference between the working electrode voltage VWE and the reference voltage VBIAS2. Combined with the negative feedback from the third node N3, the result is that the error voltage VWE−VBIAS2 becomes zero such that VBIAS2 and VWE become equal. Respective first and second currents I1, I2 are copied as respective third and fourth currents I3, I4 such that the analog output signal AO is a copy of the current IWE. The ADC 306 is thus configured as a current ADC (IADC) configured to output a digital output signal DO proportional to the current received from the second node N2.

The measurement circuit 304 of FIG. 3B has an advantage of ensuring low output impedance (when compared to the implementation in FIG. 3C) at each of the counter, reference and working electrodes CE, RE, CE, since the working electrode WE is driven directly by the first and second transistors M1, M2. Since the load across the electrochemical cell 100 is highly capacitive in nature, this inherent low output impedance may be advantageous when a stimulus of high amplitude and/or frequency is driven over the electrochemical cell 100. A drawback of the circuit 300 of FIG. 3 is that any errors in gain between the current mirror input (comprising first and second transistors M1, M2) and the current mirror output (comprising third and fourth transistors M3, M4) can lead to errors in the analog output signal AO. Such errors may include one or more of DC offset error, non-linearity, gain error and additive noise. Such errors are exacerbated at low frequencies. As such, the measurement circuit 304 tends to operate more accurately at high bandwidths. Such high bandwidths may be at frequencies up to 100 kHz, or up to 200 kHz, or up to the megahertz range.

It is common for electrochemical cells, such as the cells 100, 200 shown in FIGS. 1 and 2, to be used in power sensitive devices, such as those powered by batteries (e.g. wearable (e.g. medical) devices). In such applications, lower power consumption and efficiency of operation is advantageous.

Low power may be achieved by limiting bandwidth of measurement circuitry such as the measurement circuit 304 and ADC 306, and drive circuitry, such as the first amplifier 302. This may be acceptable when sensing analytes such as glucose or oxygen; the required signal bandwidth for measuring such analytes tends to be low, typically nearing DC levels.

Notwithstanding, in certain circumstances, it may be desirable for components of the circuit 300 to operate effectively at high bandwidths. For example, when an electrode is used in analyte sensing in a human or animal body, the electrode can decay or degrade due to reactions of the body to the presence of the electrode impacting the electrode coating. Electrode degradation can have a significant impact on impedance of the electrode and as a whole. It is desirable to be able to monitor electrode degradation by monitoring impedance of the cell. Impedance measurement involves applying high frequency, step or impulse stimuli to cell and measuring a response to that stimulus across a range of frequencies.

Examples of such techniques include electro-impedance spectroscopy (EIS) and chronoamperometry (CA).

To implement conventional EIS, the bias voltage VBIAS1 may be modulated with a sine wave and the measurement circuit 304 and ADC 306 used to measure a response of the cell 100 to that sine wave, in the form of the output voltage VO. The frequency of the sine wave may be adjusted over a range of frequencies in order to obtain a series of frequency dependent impedance measurements of the cell 100.

To implement CA, a step or impulse function stimulus is applied to the cell 100 and the measurement circuit 304 and ADC 306 used to measure a response of the cell 100 to that step or impulse function. The response can then be used to estimate or infer a transfer function between the stimulus and a response of the cell 100 to that stimulus.

In either case, impedance of a cell can be inferred from the measured response. These measurements can be repeated over time to monitor the state of health of the cell, such as the degradation of electrodes. This information can be used to compensate for electrode degradation, or to determine if an electrode has degraded to an unacceptable level, prompting a fault flag or a notification to a user to replace the electrode.

Performing accurate impedance measurements of the cell 100 typically requires that the first amplifier 302, the measurement circuit 304 and the ADC 306 are capable of operating at relatively high bandwidth, typically in the region of 10 kHz to 100 KHz. Such high-bandwidth operation tends to be at the detriment of power efficiency which, as noted above, is a key concern for battery operated wearable health sensors.

Thus, embodiments of the present disclosure aim to address or at least ameliorate one or more of the challenges discussed above and drawbacks associated with state of the art drive and measurement circuitry, such as those shown in FIGS. 3A, 3B and 3C by providing a circuitry which is operable in a low-bandwidth measurement mode and a high-bandwidth diagnostic mode.

In some embodiments, it is proposed to provide a single sensor path between the cell and the digital output comprising components which can be transitioned between the measurement mode of operation and the diagnostic mode of operation. Since the diagnostic mode is likely to consume more power than the measurement mode, due to the configuration of various components in the signal chain, circuitry may be controlled so as to periodically transition to the diagnostic mode for diagnostic measurement (e.g. impedance measurements) before returning to relatively low power measurement mode for measurements of analyte concentrations and the like.

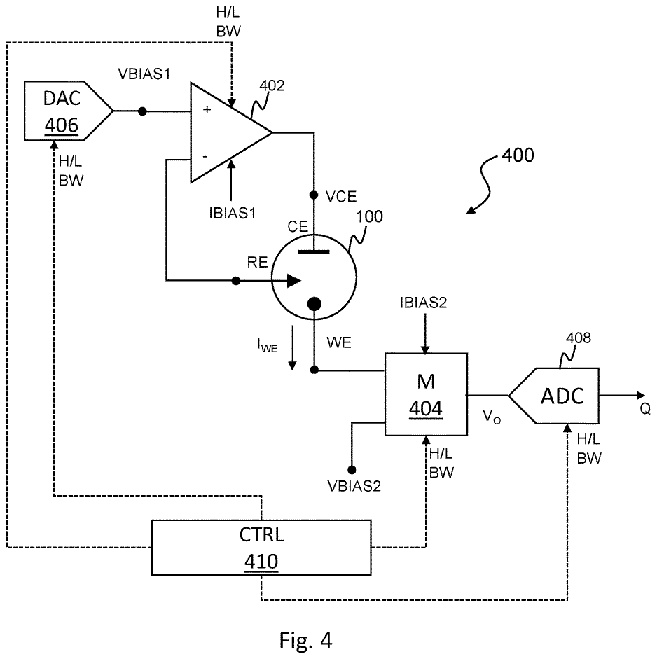

FIG. 4 illustrates an example drive and measurement circuit 400 according to embodiments of the present disclosure. Parts of the drive and measurement circuit 400 which are common to the circuits 300 of FIG. 3A have been denoted common reference numerals.

The circuit 400 comprises a first amplifier 402 and a measurement circuit 404. Each of the first amplifier 402 and the measurement circuit 404 may comprise one or more op-amps. A non-inverting input of the first amplifier 402 is coupled to a bias voltage VBIAS1 which is generated by a digital-to-analog converter (DAC) 406. An inverting input of the first amplifier 402 is coupled to the reference electrode RE. An output of the first amplifier 402 is coupled to the counter electrode CE and configured to drive the counter electrode CE with a counter electrode bias voltage VCE. The counter electrode voltage VCE applied at the counter electrode CE by the first amplifier 402 is proportional to the difference between the bias voltage VBIAS1 and the voltage VRE at the reference electrode RE.

The measurement circuit 404 is coupled between the working electrode WE and an analog-to-digital converter (ADC) 408. The measurement circuit 304 is operable to output to the ADC 408 an analog signal proportional to the current IWE flowing from the working electrode WE. The ADC 408 then converts the signal output from the measurement circuit 404 to a digital output signal Q which represents the current IWE flowing from the working electrode WE.

The circuit 400 of FIG. 4 differs from that in FIG. 3A in that each of the measurement circuit 404 and the ADC 408 are configurable between a low-bandwidth (measurement) mode and a high-bandwidth (diagnostic) mode of operation. Preferably the first amplifier 402 and the DAC 406 are also configurable in both high-and low-bandwidth modes.

The circuit 400 further comprises control circuitry 410 configured to transition the measurement circuit 404 and ADC 408 (and optionally the DAC 406 and second amplifier 402) between high-bandwidth and low-bandwidth modes. Implementation details regarding how each of the measurement circuit 404, ADC 408, DAC 406 and first amplifier 402 may be transitioned between high-bandwidth and low-bandwidth modes of operation will now be described.

The first amplifier 402 may be configurable between high-and low-bandwidth modes of operation by adjusting a level of bias current IBIAS1 that drive internal operation of the first amplifier 402. Thus, the control circuitry 410 may control the DAC 406 to adjust the bias current IBIAS1 applied to the first amplifier 402 to transition the first amplifier 402 between low-bandwidth and high-bandwidth modes of operation. Increasing the bias current IBIAS1 applied to the first amplifier 402 will increase its bandwidth. Thus, the control circuitry 410 may be configured to increase the bias current IBIAS1 applied to the first amplifier 402 to transition the first amplifier 402 from the low-bandwidth mode to the high-bandwidth mode.

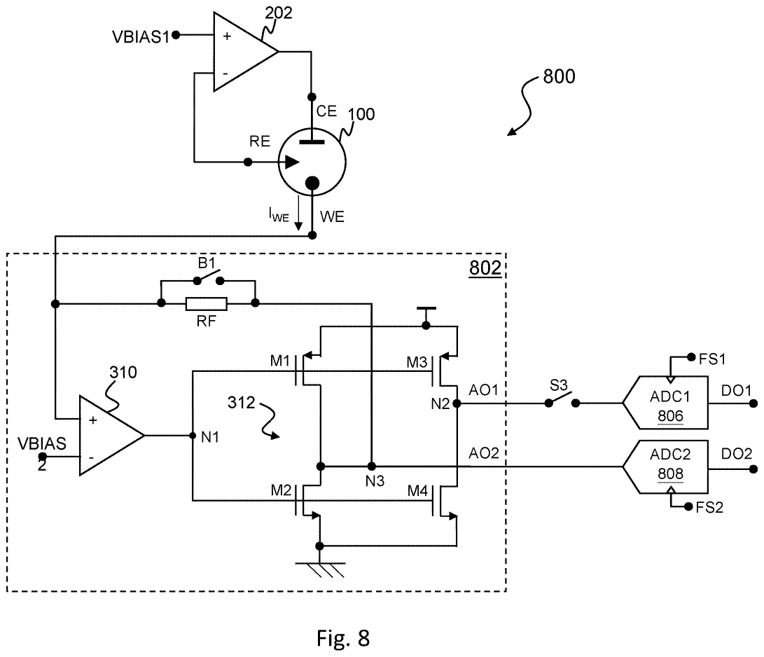

The measurement circuit 404 may comprise a current conveyor, a TIA or a combination of current conveyor and TIA, as is explained in detail below with reference to FIGS. 8 to 10. In either case, the measurement circuit 404 comprises one or more amplifier stages. As such, like the first amplifier 402, the measurement circuit 404 may also be configurable between high-and low-bandwidth modes of operation. Such configuration may be performed by adjusting a bias current IBIAS2 that drives the interla operation of the measurement circuit 404. Increasing the bias current IBIAS2 applied to the measurement circuit 404 will increase its bandwidth. Thus, the control circuitry 410 may be configured to increase the bias current IBIAS2 applied to the measurement circuit 404 to transition the measurement circuit 404 from the low-bandwidth mode to the high-bandwidth mode.

In some embodiments, the first amplifier 402, the measurement circuit 404 or both the first amplifier 402 and measurement circuit 404 are implemented as a multistage amplifier having two or more amplifier stages. Such multistage amplifiers may comprise a differential stage followed by a second stage to drive or buffer the voltage from the differential stage.

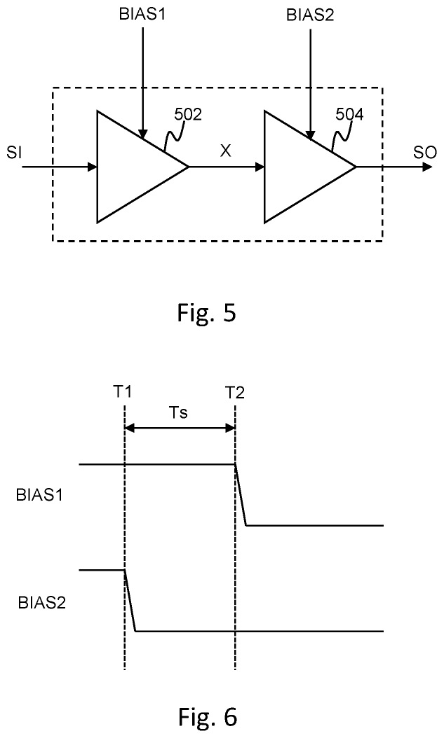

FIG. 5 is a schematic diagram of an example multistage amplifier 500 which may be implemented as the first amplifier 402 and/or in the measurement circuit 404. The multistage amplifier 500 comprises a first amplifier stage 502 and a second amplifier stage 504. The first amplifier stage 502 is configured to receive an input signal SI and output an intermediate signal X which is provided to an input of the second amplifier stage 504.

The first amplifier stage 502 may comprise a differential amplifier. In which case, the input signal SI may be a differential input signal comprising first and second input components. The first input component may be a signal of interest, and the second component may be a bias voltage.

The second amplifier stage 505 may comprise a buffer amplifier or drive amplifier. The second amplifier stage 505 may be configured to buffer or amplify the intermediate signal X received from the first amplifier stage 502 to generate the output signal SO. The second amplifier stage 505 may comprise a single ended amplifier.

The first amplifier stage 502 is biased with a first bias current BIAS1. The second amplifier stage 504 is biased with a second bias current BIAS2. The bandwidth and gain of each of the first and second amplifier stages 502, 504 may be adjusted by adjusting respective first and second bias currents BIAS1, BIAS2. Accordingly, when transitioning the circuit 400 between low-bandwidth and high-bandwidth modes of operation, the control circuitry 410 may be configured to adjust one or both of the first and second bias current BIAS1, BIAS2. Increasing one or both of the bias currents BIAS1, BIAS2 may increase the overall bandwidth of the multistage amplifier 500. Doing so will also increase the gain of the multistage amplifier 500. Conversely, decreasing one or both of the bias currents BIAS1, BIAS2 may decrease the overall bandwidth of the multistage amplifier 500. Doing so will also decrease the gain of the multistage amplifier 500.

It is advantageous to maintain a constant voltage drop between the counter electrode CE and working electrode WE of the electrochemical cell 100. However, transitions in the bias currents BIAS1, BIAS2 applied to the multistage amplifier 500 may result in transient voltages in the output signal SO. Particularly when the multistage amplifier 500 is implemented in the first amplifier 402, such transients will appear as a change in voltage drop across the cell 100 which are undesirable and may lead to prolonged errors in the cells 100 output current IWE.

When the multistage amplifier 500 is transitioned from the low-bandwidth mode to the high-bandwidth mode, voltage transients in the intermediate or output signals X, SO associated with a transition in one or both bias currents BIAS1, BIAS2 are typically attenuated by the resultant higher gain and bandwidth of the multistage amplifier 500 during and after transition to the high-bandwidth mode.

When the multistage amplifier 500 is transitioned from the high-bandwidth mode to the low-bandwidth mode, any voltage transients in the multistage amplifier 500, in particular in the intermediate signal X, may take a relatively long time to settle due to a reduction in one or both of the bias currents BIAS1, BIAS2. This is because less bias current is available to achieve settling of voltages stored on capacitive nodes within the multistage amplifier 500. This internal settling period of the multistage amplifier 500 can result in a large voltage transient in the output signal SO whose duration is determined by the time it takes for voltage nodes inside the amplifier to settle.

To reduce or substantially eliminate voltage transients in the output signal SO of the multistage amplifier 500, changes in the first and second bias currents BIAS1, BIAS2 may be made sequentially.

FIG. 6 is a timing diagram shown in example bias current sequencing regime which may be implemented during transition of the multistage amplifier 500 from the high-bandwidth mode to the low-bandwidth mode.

Firstly, at time T1, the second bias current BIAS2 may be reduced to place the second amplifier stage 504 in the low-bandwidth mode. Doing so will lead to the second amplifier stage 504 being slower and thus less charge will be kicked back from the second amplifier stage 504 onto the output of the first amplifier stage 502 (when compared to if both bias currents BIAS1, BIAS2 were switched simultaneously). During transition of the second bias current BIAS2 and for a time period Ts after that transition, the first bias current BIAS1 is maintained at its high-bandwidth mode level. In doing so, more current is provided at the output of the first amplifier stage 502 to settle any voltage transients in the intermediate signal X, thus bringing the intermediate signal to a steady state as quickly as possible.

The faster settling of the output of the first amplifier stage 502 combined with the slower response of second amplifier stage 504 (due to the reduction in second bias current BIAS2) reduces the size of any voltage transient in the output signal SO, thus improving stability of the output signal SO during transition from the high-bandwidth mode to the low-bandwidth mode.

Once the intermediate signal X at the output of the first amplifier stage 502 has settled, i.e. after the time period Ts, the first bias current BIAS1 is reduced to the low-bandwidth level, thereby transitioning the first amplifier stage 502 into the low-bandwidth mode. Reducing the first bias current BIAS1 after the intermediate signal X has settled reduces any voltage transients associated with such a bias current reduction.

Thus, by sequencing transitions in first and second bias current BIAS1, BIAS2, a substantially constant DC bias can be maintained across the electrochemical cell 100 when transitioning the multistage amplifier 500 between high-bandwidth and low-bandwidth modes.

Additionally or alternatively to sequencing transitioning of the bias current BIAS1, BIAS2, switching and drive circuitry may be provided to hold the working electrode and/or the counter electrode at a fixed voltage during transitioning of one or more elements of the circuit 400 between the low-bandwidth and high-bandwidth modes.

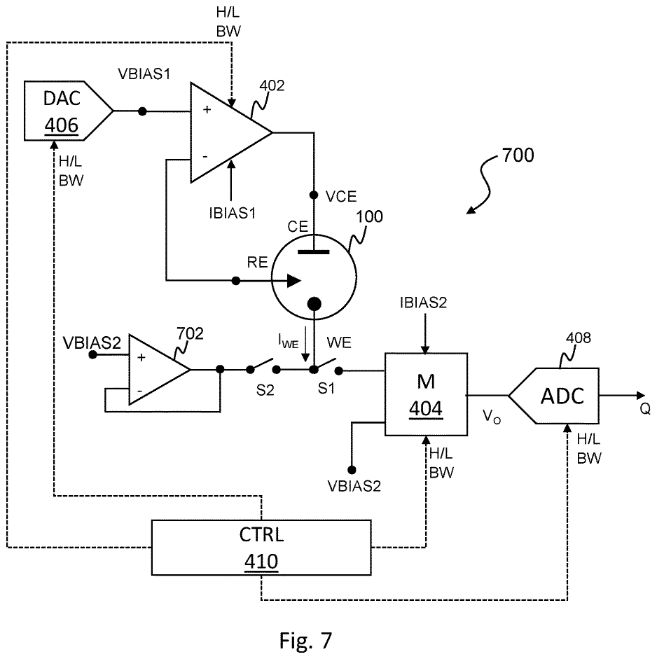

FIG. 7 is a schematic diagram of the drive and measurement circuit 700 which is a variation of the circuit 400 of FIG. 4, like parts being denoted like numberings. The circuit 700 differs from the circuit 400 of FIG. 4 by the addition of a guard amplifier 702 and first and second switches A1, A2 to allow selective isolation of the guard amplifier 702 and the measurement circuit 404 from the working electrode WE.

The first switch S1 is provided between the working electrode and the measurement circuit 404. When the first switch S1 is open, the working electrode WE is isolated from the measurement circuit 404. The second switch S2 is coupled between an output of the WE guard amplifier 702 and the working electrode WE. When the second switch A2 is open, the working electrode WE is isolated from the WE guard amplifier 702.

In the measurement mode and the diagnostic mode, the first switch S1 is closed and the second switch S2 open such that the working electrode WE is coupled to the measurement circuit 404.

During transition the circuit 700 between measurement and diagnostic modes, the first switch S1 is opened and the second switch S2 is closed, thus disconnecting the working electrode WE from the measurement circuit 404 and connecting the working electrode WE to the guard amplifier 702. The guard amplifier 702 is configured to maintain the working electrode voltage VWE at the working electrode WE at the same level as it was immediately before commencement of the transition between measurement and diagnostic modes, as well as providing a current path for the working electrode current IWE. This allows for a substantially uninterrupted bias voltage over the cell 100 during transition of the measurement circuit 404 between measurement and diagnostic modes.

Whilst not illustrated herein, a similar arrangement may optionally be provided at the counter electrode CE, to maintain the voltage VCE and current ICE at the counter electrode CE constant during transition of the first amplifier 402 between measurement and diagnostic modes.

It will also be appreciated that the guard amplifier 702 and switches S1, S2 may be used to maintain the working electrode WE in a stable condition during any period in which the input to the measurement node 404 is unstable. An example of such a situation is where autozeroing is performed on the measurement circuit 404 and the ADC 408. In such situations, a test current may be injected into the measurement circuit 404 to determine DC offset and/or gain error. In which case, it may be advantageous to isolate the measurement circuit 404 from the working electrode WE in the manner discussed above.

When adjusting the bias currents BIAS1, BIAS2 and therefore bandwidths of the multistage amplifier 500, the value of components of the amplifier 500 involved in any applicable compensation (or stability) scheme, referred to herein as compensation components, may also be adjusted. During stable operation, it is conventional to maintain a phase margin between the first and second amplifier stages 502, 504 at or around 60 degrees.

However, referring again to FIG. 6, during the transition period Ts, values of one or more of the compensation components may be adjusted to reduce capacitive loading on the first amplifier stage 502. In doing so, the rate of change of the intermediate signal X can be increased. Whilst this may result in a temporary relaxation of the phase margin during the transition period, since such relaxation is itself transient, it should not significantly interfere with the functioning of the multistage amplifier 500.

Whilst the above approaches of controlling amplifier biasing have been described with reference to multistage amplifiers, such as the multistage amplifier 500, it will be appreciated that the proposed control system may be applied to any suitable amplifier design, including those with more than two stages or just one stage.

Referring again to FIG. 4, in addition or as an alternative to adjusting the measurement circuit 404 and/or the first amplifier 402, operation of the ADC 408 and DAC 406 may also be adapted between the low-bandwidth mode and the high-bandwidth mode.

To adapt the ADC 408 for low- and high-bandwidth operation, the control circuitry 410 may be configured to adjust a sampling frequency of the ADC 408. For example, the control circuitry 410 may output a clock signal to the ADC 408 and, upon transition of the circuit 400 between the low-bandwidth mode and the high-bandwidth mode, the control circuitry 410 could adjust the frequency of the clock signal. Increasing the sampling frequency of the ADC 408 will increase the bandwidth of the ADC. For example, in the high-bandwidth mode, the control circuitry 410 may control the ADC 408 to operate with a sampling frequency of 3 MHz, and in the low-bandwidth mode, the control circuitry 410 may control the ADC 408 to operate with a sampling frequency of 32 kHz.

Additionally or alternatively to adjusting sampling frequency of the ADC 408, the control circuitry 410 may be configured to adjust a supply voltage provided to the ADC 408. Increasing the supply voltage to the ADC 408 may increase its bandwidth. Conversely, decreasing the supply voltage to the ADC 408 may decrease its bandwidth.

As noted above, the DAC 406 may be configured to generate a low-noise reference voltage VBIAS1 in the low-bandwidth, measurement, mode and a high frequency stimulus in the high-bandwidth, diagnostic, mode, to allow for high-frequency measurement of the cell 100.

As such, the control circuitry 410 may be configured to transition the DAC 406 between the low-and high-bandwidth modes of operation. To increase the bandwidth of the DAC 406, its sample rate may be increased. As required, bias currents in the DAC 406 may also need to be increased with increased sample rate.

With a move to devices that require high-bandwidth modes of operation, for example for sensor health testing, a drive and measurement circuit that is able to operate accurately and efficiently at both high and low bandwidths, rather than having to switch between separate signal paths, offers a significant advantage.

As noted above, advantages associated with the measurement circuit 404 implemented as a current conveyer are most applicable in higher bandwidth modes of operation, whereas the measurement circuit 404 implemented as a TIA is better suited for operation at lower bandwidth modes of operation. Embodiments of the present disclosure implement a hybrid design in which a low bandwidth path comprises a feedback TIA that can be reconfigured into a unity gain amplifier whose output currents are mirrored through a current conveyor stage into a high bandwidth measurement ADC.

FIG. 8 illustrates an example drive and measurement circuit 800 according to embodiments of the present disclosure. Parts of the drive and measurement circuit 800 which are common to the circuit 300 of FIG. 3C have been given common reference numerals.

Like the circuit 300, the circuit 800 comprises the first amplifier 202 having a non-inverting input coupled to a bias voltage VBIAS1 and an inverting input coupled to the reference electrode RE. The output of the first amplifier 202 is coupled to the counter electrode CE and configured to drive the counter electrode CE with a counter electrode bias voltage VCE. The counter electrode bias voltage VCE applied at the counter electrode CE by the first amplifier 202 is proportional to the difference between the bias voltage VBIAS1 and the voltage VRE at the reference electrode RE.

The drive and measurement circuit 800 further comprises a measurement circuit 802 and first and second ADCs 806, 808.

The measurement circuit 802 differs from the measurement circuit 304 shown in FIG. 3C in that a feedback resistor RF is provided in the feedback path between the third node N3 and the non-inverting input of the second amplifier 310. The feedback resistor RF is coupled between the third node N3 of the current conveyer circuitry 312 and the non-inverting input of the second amplifier 310 (and the working electrode WE of the electrochemical cell 100). In addition, a bypass switch B1 is provided in parallel with the feedback resistor RF.

The first ADC 806 has an input coupled to the second node N2. Thus, the first ADC 806 is configured to convert a first analog output signal AO1 at the second node N2 into a first digital output signal DO1. The first analog output signal AO1 is a copy of the working electrode current IWE. As such, the first ADC 806 is configured as a current ADC. Optionally, the input of the first ADC 806 may be selectively coupled to the second node N2 using a select switch S3. The first ADC 806 may be clocked by a first clock signal FS1.

The second ADC 808 has an input coupled to the third node N3. As such, the second ADC 808 is configured to convert a second analog output signal at the third node N3 into a second digital output signal which represents the signal output from the second amplifier 310. The second ADC 808 may be clocked by a second clock signal FS2. The second ADC 808 may be configured as a voltage ADC.

By operating the bypass switch S1 (and optionally the select switch S2 if provided), the measurement circuit 802 may be configured in one of two modes.

In a first mode (a current conveyor mode), the bypass switch B1 (and optionally the select switch S3) may be controlled to be closed. This causes the feedback resistor RF to be bypassed by the bypass switch B1 such that the third node N3 is coupled directly to the non-inverting input of the second amplifier 310. Since no resistance is provided in the feedback path of the second amplifier 310, the second amplifier 310 operates as a unity gain buffer. As such, when the bypass switch B1 is closed, the measurement circuit 802 operates as a current conveyor, mirroring the working electrode current IWE at the second node N2 which is coupled to input of the first (current) ADC 806 (optionally via the closed select switch S2 if provided).

In a second mode (a TIA mode), the bypass switch B1 (and optionally the select switch S3) is controlled to be open. This causes the feedback resistor RF to provide resistance in the feedback path between the output and the non-inverting input of the second amplifier 310, via the first and second transistors M1, M2. The second amplifier 310 therefore operates as a TIA converting the working electrode current IWE to a voltage (analog output signal AO2) at the third node N3. This analog output signal (voltage) AO2 is provided to the second ADC 808 which is configured as a voltage input ADC. It will be appreciated that the current output signal AO1 will still be generated in this second mode. As such, this current output signal AO1 may be used in addition to the voltage output signal AO2. Optionally, therefore, the first and second ADCs 806, 808 may therefore be operable to receive respective first and second output signal AO1, AO2 simultaneously.

It will be appreciated that since the first ADC 806 is configured to receive the first analog output signal AO1 from the second node N2 of the current conveyer 308, the first ADC 806 may be configured as a current input ADC. In an alternative embodiment, the first ADC 806 may be configured as a voltage input ADC with a TIA (not shown) provided at its input to convert the current at the second node N2 to a voltage to be provided to the first ADC 806.

It will also be appreciated that since the second ADC 806 is configured to receive the second analog output signal AO2 from the third node N3 (i.e. the output stage of the second amplifier 310 operating as a TIA), the second ADC 808 may be configured as a voltage input ADC. Alternatively, the second ADC 806 may be configured as a current ADC with a transconductance amplifier (not shown) provided at its input to convert the voltage at the third node N3 to a current to be provided to the second ADC 808.

It will be appreciated that in the second (TIA) mode, the measurement circuit 802 may more accurately process signals at low frequency when compared to the operation in the first (current conveyor) mode. In contrast, in the first (CC) mode, the measurement circuit 802 may process higher frequency signals more efficiently and/or accurately. As such, the first ADC 806 may be configured to convert signals at higher bandwidths than the second ADC 808. For example, the first clock signal FS1 used to clock the first ADC 806 may have a higher frequency than the second clock signal FS2 used to clock the second ADC 808.

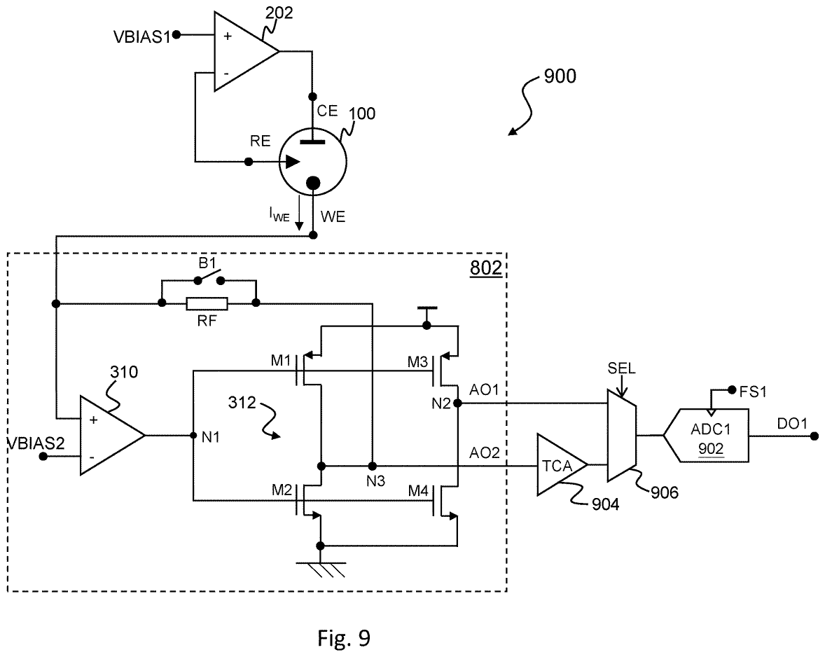

As noted above, with reference to FIG. 4, the measurement circuit 404 may be implemented using a hybrid CC/TIA arrangement, such as the arrangement shown in FIG. 8. For example, the measurement circuit 404 in FIG. 4 may comprise the measurement circuit 802 of FIG. 8. In which case, the control circuitry 410 may control switching of the bypass switch B1 and select switch S3 in the manner discussed above to transition the measurement circuit 404 between the low-bandwidth mode and the high-bandwidth mode. The first and second analog output signal AO1, AO2 may be provided to the ADC 408 via one or more multiplexers and amplifiers, such as the arrangement described below with reference to FIG. 9.

In the embodiment shown in FIG. 8, the first and second ADCs 806, 808 are provided. In other embodiments, the first and second ADCs 806, 808 may be replaced with a single ADC, for example as shown in FIG. 9.

FIG. 9 illustrates a drive and measurement circuit 900 which is a variation of the circuit 800 shown in FIG. 8, like parts having been given like numbering. The first and second ADCs 806, 808 and the select switch S3 of the circuit 800 of FIG. 8 have been replaced with an ADC 902, a transconductance amplifier (TCA) 904 and a multiplexer 906. The second node N2 is coupled to a first input of the multiplexer 906. The third node N3 is coupled to an input of the TCA 904 which converts the second analog output signal AO2 from a current to an output voltage at the TCA 904's output. The output of the TCA 904 is coupled to a second input to the multiplexer 906. An output of the multiplexer is coupled to an input of the ADC 902. The multiplexer 906 is thus configured to selectively couple one of its first and second inputs to the ADC 502 based on a select signal SEL. The ADC 902 in this embodiments is thus implemented as a current input ADC. The multiplexer 906 may be configured to switch between the first and second analog output signal paths AO1, AO2 in alignment with switching of the bypass switch S1. For example, the select signal SEL may be switched to select the first analog output AO1 when the bypass switch S1 is closed and to select the second analog output AO2 when the bypass switch S1 is open.

In a variation of the above, instead of the TCA 904 being provided between the third node N3 and the second input of the multiplexer 906, a TIA may be provided between the second node N2 and the first input of the multiplexer 906 to convert a current at the second node N2 to a voltage to be provided to the ADC 902. In which case, the ADC 902 may be implemented as a voltage input ADC.

As noted above, when the measurement circuit 404 of FIG. 4 is implemented using the measurement circuit 802 of FIGS. 8 and 9, the TCA 904 and multiplexer 906 may be provided between the measurement circuit 404 and the ADC 408 and operated in a similar manner to that described above with reference to FIG. 9 for the transition of the circuit 400 between low-and high-bandwidth modes of operation.

The hybrid solutions proposed above with reference to FIGS. 8 and 9 provides several advantages. For example, in the first mode, in which the measurement circuit 802 is operated as a current conveyor, the output impedance at the working electrode is minimized, which may be preferable for high frequency measurements. In addition, in the second mode, the TIA allows for high accuracy measurement due to the use of feedback (via the feedback resistor). Further, in both of the first and second modes, feedback can be used to set voltages at each of the counter, reference and working electrodes CE, RE, WE of the cell 100.

The hybrid architecture shown can additionally be implemented with few additional circuit elements when compared with the standard TIA architecture shown in FIG. 3B. Referring to FIG. 8, the measurement circuit 802 may be implemented with the addition of a single switch B1 to configure the second amplifier 310 as a unity gain buffer, and a current mirror (comprising the third and fourth transistors M3, M4) provided in parallel to the first and second transistors M1, M2 to serve as the output stage for the current conveyer in the first mode.

It will be appreciated that when the measurement circuit 802 is operated in the first (CC) mode, the accuracy of measurement of the properties of the cell 100 may be affected. This is due in part to the fact that the current mirror comprising the third and fourth transistors M3, M4 does not form part of the feedback path between the output and non-inverting input of the second amplifier 310. Operation of the measurement circuit 802 in this first (CC) mode can therefore lead to the addition of DC offset, gain error, distortion and/or noise.

To mitigate the effect of offset, gain error, distortion and/or noise during operation of the measurement circuit 802 in the first mode, digital correction may be implemented at the output of the first ADC 806 to correct error in the first digital output signal DO1.

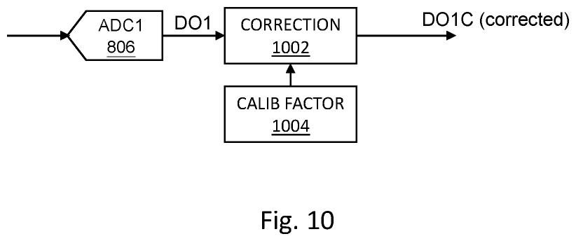

FIG. 10 illustrates an example implementation of correction of the first digital output signal DO1. The first digital output signal DO1 may be provided to a correction module 1002 configured to correct the first digital output signal DO1 and output a corrected digital output signal DO1C. The correction module 1002 may correct the first digital output signal DO1 based on a calibration factor 1004 which may be stored in memory (remote or local to the circuit 800). The calibration factor 1004 may account for one or more effects associated with operation of the measurement circuit 802 in the second (CC) mode, including but not limited to one or more of DC offset, gain error, distortion and/or noise.

The calibration factor 1004 may be obtained during a calibration process. Such a calibration process may be performed during production and/or testing of the measurement circuit 802.

When the measurement circuit 802 is operated in the first (TIA) mode, the third and fourth transistors M3, M4 are not used to generate the first digital output signal DO1. Instead, the first and second transistors M1, M2, which are in the feedback loop of the second amplifier 310, are used to generate the first digital output signal DO1. Because of this, the first digital output signal DO1 (or the first analog output signal AO1) can be used as a reference in the calibration process.

Referring, for example, to FIG. 8, the circuit 800 may be stimulated with appropriate input stimuli. Such stimuli may be applied, for example, at the working electrode WE. The input stimuli may comprise of DC signals (constant and/or swept) or some combination of DC and AC signals. Such input stimuli may be applied to the circuit 400 during the calibration process.

The first and second digital output signal DO1, DO2 may be compared to determine an error in the first digital output signal DO1 at various input signal conditions (thereby using the second digital output signal DO2 as a reference). Such errors can then be used to generate the calibration factor 1004 which may be frequency and time dependent.

Additionally or alternatively, a calibration process may use a signal at the working electrode WE during normal operation of the circuit 800. Realtime comparison of the first and second digital output signals DO1, DO2 may be used to continuously correct for errors in the first digital output signal substantially continuously or periodically to ensure that the corrected first digital output signal remains accurate during operation of the measurement circuit 802.

Embodiments above are described with reference to a three-electrode cell 100 comprising a counter electrode CE, a working electrode WE and a reference electrode RE. Embodiments of the disclosure are not, however, limited to having three-electrodes. The concepts described herein are equally applicable to two-electrode cells. In particular, in any of the embodiments described above, the three-electrode cell 100 may be replaced with the two-electrode cell 200 shown in FIG. 2.

Embodiments are described above with reference to cells 100, 200 comprising a single counter electrode CE and a single working electrode WE. Embodiments of the disclosure are not, however, limited to having cells having only one counter electrode or only one working electrode. The concepts described herein are equally applicable to cells comprising multiple working electrodes or multiple counter electrodes. In doing so, such sensors may either be providing redundancy or enabling the sensing of multiple analytes in a single chip. This may be particularly advantageous in applications such as continuous glucose monitoring, where it may be desirable to measure concentrations of several analytes including but not limited to two or more of glucose, ketones, oxygen, lactate, and the like.

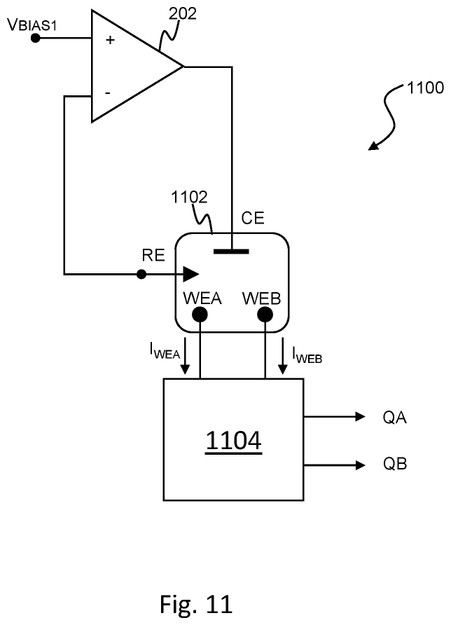

FIG. 11 illustrates an example drive and measurement circuit 1100. Where like parts have been given like numbering. In the circuit 1100, an electrochemical cell 1102 comprising first and second working electrode WEA, WEB, a counter electrode CE, and a reference electrode RE. A measurement circuit 1104 is provided which outputs a first digital output signals QA based on a current IWEA derived from the first working electrode WEA and outputs a second digital output signal QB based on a current IWEB derived from the second working electrode WEB. The measurement circuit 1104 may, for example, comprise two processing channels, each processing channel implementing the circuitry shown in FIG. 4, 8 or 9. Alternatively, the measurement circuit may be implemented using a single processing stream multiplexed by a multiplexer (not shown). In either case, the measurement circuit 1104 may be operable to hold the first and second working electrodes WEA, WEB at different voltages. This may be particularly useful when measuring different analytes with the first and second working electrodes WEA, WEB.

It will be appreciated that, whilst the embodiment described comprises two working electrodes WEA, WEB, in other embodiments three or more counter electrodes may be provided.

In the embodiments described herein, the electrochemical cells 100, 200, 1102 have been described in the form of an electrochemical sensor comprising counter and working electrodes CE, WE. For such sensors, the stimulus is typically a voltage, and the measured response is a current. It will be appreciated that embodiments of the present disclosure are not limited to such cells and extend to other types of cells, such as electrochemical cells acting as a power source (i.e. a battery). For batteries and the like, the driving stimulus of the cell is typically a current, and the measured response a voltage.

The various circuitry and electrochemical cells described herein may be incorporated into a continuous analyte sensor or a continuous glucose sensor or a continuous glucose monitor. The terms “continuous analyte sensor”, “continuous glucose sensor”, and “continuous glucose monitor” as used herein, will be well-known to a person of ordinary skill in the art and are not to be limited to a special or customized meaning. These terms refer, without limitation, to a device that continuously measures a concentration of an analyte/glucose and/or calibrates the sensor or an electrochemical cell incorporated therein (e.g., by continuously adjusting or determining the sensor's sensitivity and background).

The skilled person will recognise that some aspects of the above-described apparatus and methods may be embodied as processor control code, for example on a non-volatile carrier medium such as a disk, CD-or DVD-ROM, programmed memory such as read only memory (Firmware), or on a data carrier such as an optical or electrical signal carrier. For many applications embodiments of the invention will be implemented on a DSP (Digital Signal Processor), ASIC (Application Specific Integrated Circuit) or FPGA (Field Programmable Gate Array). Thus the code may comprise conventional program code or microcode or, for example code for setting up or controlling an ASIC or FPGA. The code may also comprise code for dynamically configuring re-configurable apparatus such as re-programmable logic gate arrays. Similarly the code may comprise code for a hardware description language such as Verilog TM or VHDL (Very high speed integrated circuit Hardware Description Language). As the skilled person will appreciate, the code may be distributed between a plurality of coupled components in communication with one another. Where appropriate, the embodiments may also be implemented using code running on a field-(re)programmable analogue array or similar device in order to configure analogue hardware.

Note that as used herein the term module shall be used to refer to a functional unit or block which may be implemented at least partly by dedicated hardware components such as custom defined circuitry and/or at least partly be implemented by one or more software processors or appropriate code running on a suitable general purpose processor or the like. A module may itself comprise other modules or functional units. A module may be provided by multiple components or sub-modules which need not be co-located and could be provided on different integrated circuits and/or running on different processors.

Embodiments may be implemented in a host device, especially a portable and/or battery powered host device such as a mobile computing device for example a laptop or tablet computer, a games console, a remote control device, a home automation controller or a domestic appliance including a domestic temperature or lighting control system, a toy, a machine such as a robot, an audio player, a video player, or a mobile telephone for example a smartphone. Embodiments may be implemented in a wearable or implanted host device, such as a continuous glucose monitor or the like.

As used herein, when two or more elements are referred to as “coupled” to one another, such term indicates that such two or more elements are in electronic communication or mechanical communication, as applicable, whether connected indirectly or directly, with or without intervening elements.

This disclosure encompasses all changes, substitutions, variations, alterations, and modifications to the example embodiments herein that a person having ordinary skill in the art would comprehend. Similarly, where appropriate, the appended claims encompass all changes, substitutions, variations, alterations, and modifications to the example embodiments herein that a person having ordinary skill in the art would comprehend. Moreover, reference in the appended claims to an apparatus or system or a component of an apparatus or system being adapted to, arranged to, capable of, configured to, enabled to, operable to, or operative to perform a particular function encompasses that apparatus, system, or component, whether or not it or that particular function is activated, turned on, or unlocked, as long as that apparatus, system, or component is so adapted, arranged, capable, configured, enabled, operable, or operative. Accordingly, modifications, additions, or omissions may be made to the systems, apparatuses, and methods described herein without departing from the scope of the disclosure. For example, the components of the systems and apparatuses may be integrated or separated. Moreover, the operations of the systems and apparatuses disclosed herein may be performed by more, fewer, or other components and the methods described may include more, fewer, or other steps. Additionally, steps may be performed in any suitable order. As used in this document, “each” refers to each member of a set or each member of a subset of a set.

Although exemplary embodiments are illustrated in the figures and described below, the principles of the present disclosure may be implemented using any number of techniques, whether currently known or not. The present disclosure should in no way be limited to the exemplary implementations and techniques illustrated in the drawings and described above.

Unless otherwise specifically noted, articles depicted in the drawings are not necessarily drawn to scale.

All examples and conditional language recited herein are intended for pedagogical objects to aid the reader in understanding the disclosure and the concepts contributed by the inventor to furthering the art, and are construed as being without limitation to such specifically recited examples and conditions. Although embodiments of the present disclosure have been described in detail, it should be understood that various changes, substitutions, and alterations could be made hereto without departing from the spirit and scope of the disclosure.

Although specific advantages have been enumerated above, various embodiments may include some, none, or all of the enumerated advantages. Additionally, other technical advantages may become readily apparent to one of ordinary skill in the art after review of the foregoing figures and description.

It should be noted that the above-mentioned embodiments illustrate rather than limit the invention, and that those skilled in the art will be able to design many alternative embodiments without departing from the scope of the appended claims. The word “comprising” does not exclude the presence of elements or steps other than those listed in a claim, “a” or “an” does not exclude a plurality, and a single feature or other unit may fulfil the functions of several units recited in the claims. Any reference numerals or labels in the claims shall not be construed so as to limit their scope.

1. Circuitry for processing an analyte signal obtained from an electrochemical cell comprising a first electrode and a second electrode, the circuitry comprising:
drive circuitry comprising a drive amplifier configured to apply a stimulus to the first electrode;
measurement circuitry comprising a measurement amplifier configured to measure a response at the second electrode of the electrochemical cell to the stimulus;
an analog-to-digital converter (ADC) configured to output a digital output in dependence on the measured response; and
control circuitry operable to transition the circuitry between a measurement mode and a diagnostic mode, wherein:
in the measurement mode, the measurement circuitry is configured with a first bandwidth for measuring an analyte at the second electrode of the cell; and
in a diagnostic mode the measurement circuitry is configured with a second bandwidth for measuring an impedance of the second electrode of the cell, wherein the second bandwidth is greater than the first bandwidth.

2. Circuitry of claim 1, wherein the control circuitry is configured to transition the measurement circuitry between the measurement mode and the diagnostic mode by adjusting a first bias signal applied to the measurement amplifier.

3. Circuitry of claim 1, wherein one or both of the measurement amplifier and the drive amplifier comprises a multistage amplifier comprising:
a first amplifier stage comprising a first stage input and a first stage output, the first amplifier stage biased with a first bias signal; and
a second amplifier stage having a second stage input coupled to first stage output and a second stage output, the second amplifier stage biased with a second bias signal,
wherein transitioning the circuitry between the measurement mode and the diagnostic mode comprises:
adjusting the first bias signal and the second bias signal.

4. Circuitry of claim 3, wherein the first and second bias signals are adjusted to maintain a constant DC bias at the output of the second amplifier stage.

5. Circuitry of claim 3, wherein transitioning the circuitry from the diagnostic mode to the measurement mode comprises:
during a first time period, reducing a level of the second bias signal; and
in a second time period, reducing a level of the first bias signal, the second time period occurring after the first time period.

6. Circuitry of claim 5, wherein the first and second time period are separated by an intermediate time period, wherein during the intermediate time period, the control circuitry is configured to adjust a compensation scheme of the multistage amplifier.

7. (canceled)

8. Circuitry of claim 5, wherein the compensation scheme is adjusted to increase a response of the first amplifier stage during the intermediate time period.

9. Circuitry of claim 3, wherein transitioning the circuitry from the measurement mode to the diagnostic mode comprises:
increasing a level of the second bias signal; and
increasing a level of the first bias signal, the levels of the first and second bias signals increased substantially simultaneously.

10. Circuitry of claim 1, wherein the measurement circuitry comprises:
a first signal path between the second electrode of the electrochemical cell and a first input of the ADC, the first signal path comprising a first gain stage configured to convert the analyte signal to a first analog signal;
a second signal path between the first electrode and the first input of the ADC, the second signal path comprising a second gain stage configured to convert the analyte signal to a second analog signal, first gain stage having a higher bandwidth than the second gain stage; and
switching circuitry configured to selectively couple the second electrode to the first input of the ADC;
wherein the control circuitry is configured to control the switching circuitry to couple the second electrode to the first input of the ADC to transition the circuitry from the measurement mode to the diagnostic mode.

11. Circuitry of claim 10, wherein:
the first gain stage comprises a current conveyor and the second gain stage comprises a transimpedance amplifier (TIA) comprising a feedback resistor coupled between the second electrode and the first input of the ADC,
wherein selectively coupling the first electrode to the first input of the ADC circuit comprises bypassing the feedback resistor.

12. Circuitry of claim 1, wherein:
in the measurement mode, the drive circuitry is configured with the first bandwidth; and
in the diagnostic mode, the drive circuitry is configured with the second bandwidth.

13. Circuitry of claim 12, wherein the control circuitry is operable to transition the measurement circuitry between the measurement mode and the diagnostic mode by adjusting a second bias signal applied to the drive amplifier.

14. Circuitry of claim 13, wherein the drive circuitry comprises a digital-to-analog converter (DAC) configured to apply the second bias signal to the drive circuitry.

15. Circuitry of claim 14, wherein the DAC is configurable in the measurement mode and the diagnostic mode, wherein configuring the DAC between the measurement mode and the diagnostic mode comprises adjusting a sampling rate of the DAC.

16. (canceled)

17. Circuitry of claim 1, wherein, the ADC is configurable in the measurement mode and the diagnostic mode, wherein configuring the ADC between the measurement mode and the diagnostic mode comprises adjusting a sampling frequency of the ADC or an ADC supply voltage provided to the ADC, wherein the sampling frequency in the measurement mode is lower than the sampling frequency in the diagnostic mode.

18. (canceled)

19. Circuitry of claim 1, wherein in the measurement mode, the stimulus comprises a DC component, and wherein in the diagnostic mode, the stimulus comprises the DC component and an AC component.

20. Circuitry of claim 19, wherein the AC component of the stimulus comprises a sinusoid, a step function, or an impulse function.

21. Circuitry of claim 1, wherein, in the measurement mode, the circuitry is configured to determine an analyte concentration of the analyte at the second electrode.

22. Circuitry of claim 1, wherein, in the diagnostic mode, the circuitry is configured to determine a status of the second electrode in dependence on the measured impedance, wherein the status comprises one or more of the following:
a state of health of the electrochemical cell;
a state of degradation of the second electrode; and
a fault at the electrochemical cell.

23. (canceled)

24. Circuitry of claim 1, wherein the control circuitry is operable to configure the circuitry in the measurement mode or the diagnostic mode in response to a command from an external controller.

25. Circuitry of claim 1, wherein the control circuitry is operable to transition the circuitry between the measurement mode and the diagnostic mode after a predetermined time period since a last transition between the measurement mode and the diagnostic mode.

26. Circuitry for processing an analyte signal obtained from an electrochemical cell comprising a first electrode and a second electrode, the circuitry comprising:
drive circuitry comprising a drive amplifier configured to apply a stimulus to a first electrode;
measurement circuitry comprising a measurement amplifier configured to measure a response at the second electrode;
wherein one or both of the measurement amplifier and the drive amplifier comprises a multistage amplifier comprising:
a first amplifier stage comprising a first stage input and a first stage output, the first amplifier stage biased with a first bias signal; and
a second amplifier stage having a second stage input coupled to first stage output and a second stage output, the second amplifier stage biased with a second bias signal.

27. Circuitry of claim 26, further comprising control circuitry operable to transition the circuitry between a measurement mode and a diagnostic mode, wherein
transitioning the circuitry between the measurement mode and the diagnostic mode comprises adjusting the first bias signal and the second bias signal.

28.-46. (canceled)

47. A system comprising:
the circuitry of claim 1;
the electrochemical cell.

48. The system of claim 47, wherein:
the electrochemical cell comprising a counter electrode;
the first electrode is a first working electrode of the electrochemical cell.

49. The system of claim 48, wherein the electrochemical cell comprises one or more a second working electrodes.

50. The system of claim 48, wherein the electrochemical cell comprises an anode and a cathode, wherein the first electrode is the cathode.

51. An integrated circuit comprising the circuitry of claim 1.

52. An electronic device comprising the circuitry of claim 1, wherein the electronic device comprises one or more of a wearable health monitor, an analyte sensor, a continuous glucose monitor, a battery, a mobile computing device, a laptop computer, a tablet computer, a games console, a remote control device, a home automation controller or a domestic appliance, a toy, a robot, an audio player, a video player, or a mobile telephone, and a smartphone.

53. (canceled)
